(19) United States
(12) Patent Application Publication
(10) Pub. No.: US 2013/0253271 A1
(43) Pub. Date: Sep. 26, 2013

(54) ENDOSCOPE

(71) Applicants: Hee Moon Jeong, Yongin-si (KR); Jin Ho Lee, Suwon-si (KR); Kae Dong Back, Yongin-si (KR)

(72) Inventors: Hee Moon Jeong, Yongin-si (KR); Jin Ho Lee, Suwon-si (KR); Kae Dong Back, Yongin-si (KR)

(73) Assignee: SAMSUNG ELECTRONICS CO., LTD., Suwon-si (KR)

(21) Appl. No.: 13/989,616

(22) PCT Filed: Nov. 2, 2011
(86) PCT No.: PCT/KR2011/008298

§ 371 Date: May 24, 2013

(30) Foreign Application Priority Data
Nov. 24, 2010 (KR) 10-2010-0117368

(51) Int. Cl. A61B 1/005

(52) U.S. Cl. CPC A61B 1/0052; A61B 1/0055
USPC 600/137

(57) ABSTRACT

Disclosed is an endoscope having a rotating device to axially rotate a bending member. The endoscope includes a flexible member which is freely bendable upon receiving external force, a bending member provided at a front end of the flexible member, the bending member being bendable with one degree of freedom, a rotating device rotatably coupled to the flexible member in an axial direction of the flexible member and serving to rotate the bending member in the axial direction, and an operating unit connected to the rotating device and having a rotation operation unit to rotate the rotating device.

### CROSS-REFERENCE TO RELATED APPLICATIONS

This application is a National Stage application under 35 U.S.C. §371 of International Application No. PCT/KR2011/008298, filed on Nov. 2, 2011, which claims priority to Korean Application No. 10-2010-0117368, filed Nov. 24, 2010, the disclosure of each is incorporated herein by reference.

### TECHNICAL FIELD

Embodiments of the present invention relates to an endoscope having a rotating device to axially rotate a bending member.

### BACKGROUND ART

Generally, a medical endoscope is a medical instrument which is inserted into a body cavity or an internal organ to allow an operator to view the interior of the internal organ without a surgical operation and has made considerable contributions to medicine.

Unlike other medical imaging instruments such as radiography systems, an endoscope is directly inserted into the human body. Thus, endoscopic inspection advantageously allows an operator to directly observe the shape or condition of an internal organ or a cavity of the human body with the naked eye.

Such an endoscope includes a bending member which is bendable to move a view member at a front end of the endoscope within the human body, and an operating unit which an operator grips to operate the bending member.

The bending member has two degrees of freedom (2-DOF) so as to bend in a vertical direction and a horizontal direction. Bending in the vertical direction and bending in the horizontal direction are performed independently of each other. As such, an operator moves the view member at the front end of the endoscope to a target position by bending the bending member in the vertical direction or the horizontal direction while gripping the operating unit.

However, although bending in the vertical direction and bending in the horizontal direction are performed independently of each other, if bending motions in the two directions are coupled with each other, an actual bending output differs from a bending value input by an operator using the operating unit. This may problematically necessitate several bending operations to move the view member at the front end of the endoscope to a target position.

### DISCLOSURE

### Technical Problem

It is an aspect of the present invention to provide an endoscope having a rotating device to axially rotate a bending member.

### Technical Solution

In accordance with one aspect of the present invention, an endoscope includes a flexible member which is freely bendable upon receiving external force, a bending member provided at a front end of the flexible member, the bending member being bendable with one degree of freedom, a rotating device rotatably coupled to the flexible member in an axial direction of the flexible member and serving to rotate the bending member in the axial direction, and an operating unit connected to the rotating device and having a rotation operation unit to rotate the rotating device.

The bending member may include a plurality of bending segments jointed to one another and elastic members placed between the respective bending segments to return the plurality of bending segments to original positions thereof.

At least a part of the flexible member may undergo torsion when axial torque is applied to the flexible member, and the bending member may be secured to the front end of the flexible member and is rotatable along with the flexible member.

The rotating device may include a torsional device coupled to the flexible member so as to apply the axial torque to the flexible member, and the flexible member may include a first flexible portion secured to the operating unit, a second flexible portion connected to a front end of the first flexible portion so as to undergo torsion when the torque is applied thereto, and a third flexible portion connected to a front end of the second flexible portion so as to be rotated in the axial direction.

The torsional device may include a coupling member secured to the operating unit and the first flexible portion so as to secure the first flexible portion to the operating unit, and a rotating member rotatably coupled to the coupling member while being secured to the third flexible portion.

The coupling member may include a rotation guide boss formed on a circumference thereof, and the rotating member may include a rotation guide recess into which the rotation guide boss is inserted.

The rotating member may include a rotating body secured to the third flexible portion to extend toward the first flexible portion, and a rotating disc secured to the rotating body and adapted to receive the torque from the rotation operation unit.

The torsional device may further include a friction member placed between the coupling member and the rotating member to increase friction.

The rotation operation unit may include a rotation wire connected to the rotating device, the rotating device may include a rotating tube assembly formed by axially connecting at least two tubes to each other, and a front end of the rotating tube assembly may be axially rotatable relative to a rear end of the rotating tube assembly.

The rotating tube assembly may include a stationary tube secured to the flexible member and a rotating tube rotatably inserted into the stationary tube while being secured to the bending member.

The stationary tube may include a rotation guide slit perforated in a circumference thereof, the rotating tube may include a rotation pin movably inserted into the rotation guide slit, and the rotating tube may be rotatable relative to the stationary tube as the rotation pin is moved along the rotation guide slit.

The rotating tube may further include a rotation wire connector to which the rotation wire is secured.

The rotation wire connector may be secured to the rotation pin.

The stationary tube may further include a rotation wire hanger to guide the rotation wire to the rotation wire connector.

The rotating tube assembly may further include a sliding tube coupled to the stationary tube so as to reciprocate in an axial direction of the stationary tube.

The stationary tube may include a reciprocation guide pin to guide reciprocation of the sliding tube, and the sliding tube may include a reciprocation guide slot into which the reciprocation guide pin is inserted.

The sliding tube may include a rotation leading slit perforated in a curved circumferential wall thereof to extend obliquely relative to a center axis of the sliding tube, the rotating tube may include a rotation pin inserted into the rotation leading slit, and the rotating tube may be rotated during reciprocation of the sliding tube as the rotation pin is moved along the rotation leading slit.

The stationary tube may further include a rotation guide groove to guide rotation of the rotating tube, and the rotating tube may further include a rotation protrusion circumferentially formed and inserted into the rotation guide groove.

In accordance with another aspect of the present invention, an endoscope includes
a flexible member that is bendable in an arbitrary direction, a bending member connected to a front end of the flexible member, the bending member being bendable with one degree of freedom, an operating unit for an operator to grip placed behind the flexible member, and a rotating device coupled to the flexible member so as to be rotated in an axial direction of the flexible member and serving to rotate the bending member in the axial direction, wherein the operating unit includes a housing defining an external appearance of the operating unit, a bending operation unit mounted to the housing and connected to the bending member to enable bending of the bending member, and a rotation operation unit mounted to the housing and connected to the rotating device, and wherein the bending operation unit includes a bending wire connected to the bending member.

A rear end of the flexible member may be secured to an inner position of the housing, the rotating device may include a torsional device coupled to the flexible member to surround at least a part of the flexible member so as to transmit axial torque to the flexible member, and the torsional device may be placed within the housing.

The bending operation unit may further include a bending operation pulley placed within the housing to operate the bending wire, the rotation operation unit may include a rotation wire connected to the rotating device and a rotation operation pulley placed within the housing to operate the rotation wire, and the bending operation pulley and the rotation operation pulley may have the same axis of rotation.

The bending operation unit may further include a bending operation knob rotatably mounted to the housing to operate the bending wire, the rotation operation unit may include a rotation wire connected to the rotating device and a rotation operation knob rotatably mounted to the housing to operate the rotation wire, and an axis of rotation of the bending operation unit and an axis of rotation of the rotation operation knob may be perpendicular to each other.

The rotation operation unit may include a rotation wire connected to the rotating device, and the bending wire and the rotation wire may be arranged close to each other within the flexible member.

In accordance with another aspect of the present invention, an endoscope includes
a flexible member that is bendable in an arbitrary direction, a bending member connected to a front end of the flexible member, the bending member being bendable with one degree of freedom, a torsional device coupled to the flexible member to transmit axial torque to the flexible member, and an operating unit including a rotation operation unit connected to the torsional device, wherein the flexible member includes a first flexible portion secured to the operating unit, a second flexible portion connected to a front end of the first flexible portion so as to undergo torsion when the torque is applied thereto, and a third flexible portion connected to a front end of the second flexible portion so as to be rotated in the axial direction, and wherein the torsional device includes a coupling member secured to the operating unit and the first flexible portion so as to secure the first flexible portion to the operating unit, and a rotating member rotatably coupled to the coupling member while being secured to the third flexible portion.

### Advantageous Effects

With provision of a bending member having one degree of freedom and performing axial rotation, motion of the bending member may correspond to manipulation of an operator. Thus, the bending member is bent and axially rotated according to operator manipulation, which allows the operator to more easily move a view member of an endoscope to a target position.

In addition, easy movement of the view member of the endoscope may reduce time required for endoscopic inspection, which may reduce physical burden applied to a person being diagnosed.

### DESCRIPTION OF DRAWINGS

These and/or other aspects of the invention will become apparent and more readily appreciated from the following description of the embodiments, taken in conjunction with the accompanying drawings of which:

FIG. 1 is a view illustrating an endoscope system according to an embodiment of the present invention;

FIG. 2 is a view illustrating an insert unit of an endoscope according to an embodiment of the present invention;

FIGS. 3 and 4 are views illustrating a bending member of the endoscope according to one embodiment of the present invention;

FIG. 5 is a perspective view illustrating a rotating device according to an embodiment of the present invention;

FIG. 6 is a cross sectional view illustrating a configuration of the rotating device;

FIG. 7 is a view illustrating an operation of the rotating device;

FIG. 8 is a perspective view illustrating a rotating device according to another embodiment of the present invention;

FIG. 9 is an exploded perspective view illustrating major components of the rotating device;

FIGS. 10 to 12 are views illustrating an operation of the rotating device;

FIG. 13 is a perspective view illustrating a configuration of a rotating device according to a further embodiment of the present invention;

FIG. 14 is an exploded perspective view illustrating major components of the rotating device;

FIGS. 15 to 17 are views illustrating an operation of the rotating device; and

FIGS. 18 and 19 are views illustrating arrangement of a rotation wire according to an embodiment of the present invention.

### BEST MODE

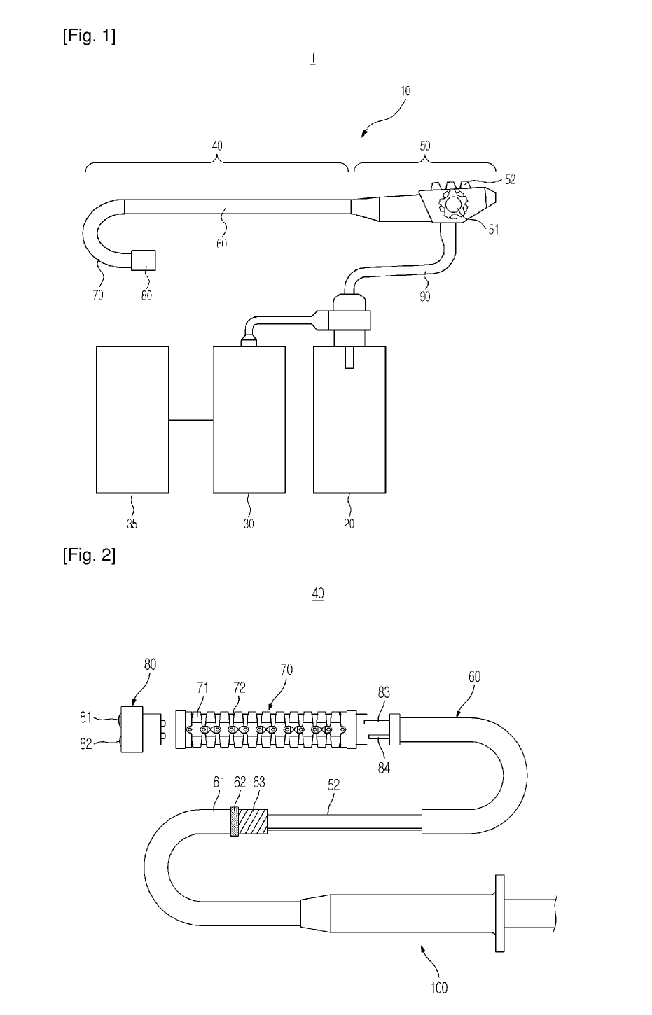

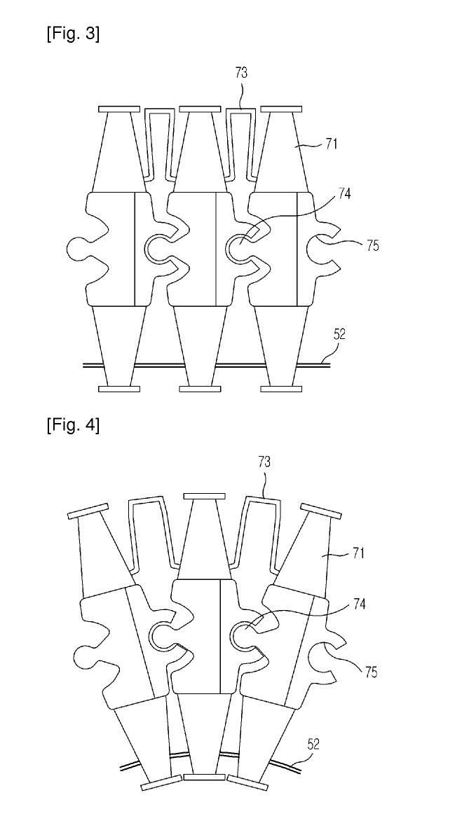

Reference will now be made in detail to the exemplary embodiments of the present invention, examples of which are illustrated in the accompanying drawings, wherein like reference numerals refer to like elements throughout. FIG. 1 is a view illustrating an endoscope system according to an embodiment of the present invention, FIG. 2 is a view illustrating an insert unit of an endoscope according to an embodiment of the present invention, and FIGS. 3 and 4 are views illustrating a bending member of the endoscope according to one embodiment of the present invention.

As illustrated in FIGS. 1 and 2, an endoscope system 1 for use in endoscopic inspection includes an endoscope 10, a light source device 20, a signal processing device 30, and a display device 35. The endoscope 10 includes an insert unit 40 to be inserted into an internal organ or a body cavity, and an operating unit 50 that an operator grips to perform a variety of functions of the endoscope 10.

The insert unit 40 includes a freely bendable flexible member 60, a bending member 70, and a hard tip member 80 connected to a front end of the bending member 70. The flexible member 60 is connected to the operating unit 50. The flexible member 60 may consist of a helical steel band 63, a metallic wire mesh 62 surrounding the helical steel band 63, and a covering tube 61. The wire mesh 62 may be made of stainless steel. The entire flexible member 60 has sufficient flexibility to be passively bent upon receiving external force.

The tip member 80 may be provided with an objective lens 81 to focus on an object and a light guide lens 82 to guide light directed from the light source device 20 to the object. A charge coupled device (not shown) may be provided behind the objective lens 81 to convert an image of the object collected by the objective lens 81 into an image signal. The charge coupled device may be connected to the objective lens 81.

A signal cable 83 is connected to the charge coupled device (not shown) and is inserted through the insert unit 40 and the operating unit 50. The signal cable 83 is also connected to the signal processing device 30 by way of the interior of a universal cable 90 that extends rearward from the operating unit 50.

A light guide 84 is inserted through the insert unit 40 and the operating unit 50 to guide light from the light source device 20 to the object. The light guide 84 is connected to the light source device 20 by way of the interior of the universal cable 90. The light source device 20 irradiates light into the human body through the light guide 84.

The signal processing device 30 receives an image photographed by the tip member 80 of the endoscope 10 through the signal cable 83 and processes the image signal. The display device 35 receives the image signal from the signal processing device 30 and displays it to the operator of the endoscopy system.

The bending member 70 is connected to a front end of the flexible member 60. The bending member 70 takes the form of a tube consisting of a plurality of ring-shaped bending segments 71 connected to one another while defining a space therein. The plurality of bending segments 71 is centrally jointed to one another such that the bending member 70 is bendable with one degree of freedom. The respective bending segments 71 may be provided at a coupling portion thereof with a connector, such as a rivet or the like. The bending member 70 is bendable within a single plane.

A bending wire 52 is inserted through the bending member 70. More particularly, a pair of bending wires 52 is provided. These bending wires 52 may be arranged to face each other within the interior space of the bending member 70. One end of each bending wire 52 is secured to a front end of the bending member 70 and the other end of the bending wire 52 sequentially passes through the bending member 70 and the flexible member 60 to thereby be connected to the operating unit 50. The operating unit 50 is provided with a bending operation unit 51 connected to the bending wires 52. As the operator pulls any one of the pair of bending wires 52 by operating the bending operation unit 51, the bending member 70 is bendable within a single plane.

As illustrated in FIGS. 3 and 4, the bending member 70 includes the plurality of bending segments 71 and a plurality of elastic members 73. The single bending wire 52 is inserted through the bending member 70 and the elastic member 73 is arranged at an opposite side of the bending wire 52. Each bending segment 71 includes a first joint 74 and a second joint 75 shaped to correspond to each other, whereby the plurality of bending segment 71 is coupled to one another without separate connecting members.

The elastic members 73 are placed between the respective bending segments 71. One end of the single bending wire 52 is connected to the front end of the bending member 70 and the other end of the bending wire 52 is connected to the bending operation unit 51. If the operator applies tension to the bending wire 52 by operating the bending operation unit 51, elastic energy is accumulated in the elastic member 73 simultaneously with bending of the bending member 70. Then, if the operator releases the tension applied to the bending wire 52, the bending segments 71 are returned to original positions thereof by the elastic energy of the elastic member 73.

As the bending member 70 is bendable within a single plane, a viewing range of the endoscope 10 is limited to the plane in which the bending member 70 is bendable. Accordingly, the endoscope 10 includes a rotating device 100, which axially rotates the bending member 70 so as to move a view member of the endoscope 10 to various positions within the human body.

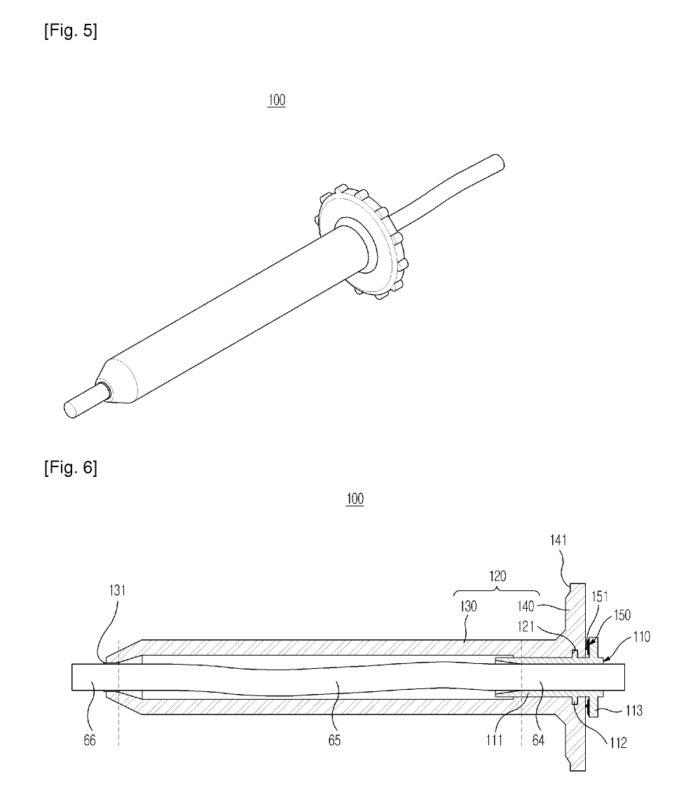

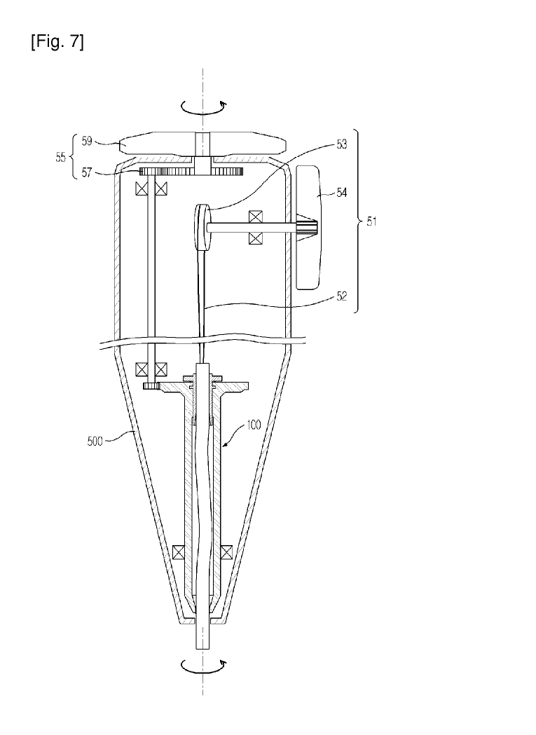

FIG. 5 is a perspective view illustrating the rotating device according to an embodiment of the present invention, FIG. 6 is a cross sectional view illustrating a configuration of the rotating device, and FIG. 7 is a view illustrating an operation of the rotating device.

As illustrated in FIGS. 5 to 7, the rotating device includes a torsional device 100 coupled to the flexible member 60. The torsional device 100 is coupled to the flexible member 60 so as to surround the flexible member 60, thereby applying axial torque to the flexible member 60.

The flexible member 60 includes a first flexible portion 64 secured to the operating unit 50, a second flexible portion 65 connected to a front end of the first flexible portion 64, the second flexible portion 65 performing torsional motion, and a third flexible portion 66 connected to a front end of the second flexible portion 65 so as to be axially rotated.

To allow the torsional device 100 to perform torsional motion upon receiving torque, the second flexible portion 65 may have lower torsional rigidity than the first flexible portion 64 and the third torsional portion 66. To this end, the second flexible portion 65 may be made of a helical metallic band or wire mesh having a small torsional rigidity, or may not contain the aforementioned metallic structures.

The torsional device 100 includes a coupling member 110 secured to the operating unit 50 and the first flexible portion 64, a rotating member 120 secured to the third flexible portion 66 while being axially rotatably coupled to the coupling member 110, and a friction member 150 placed between the coupling member 110 and the rotating member 120.

The coupling member 110 has a hollow cylindrical shape such that the first flexible portion 64 is inserted through the coupling member 110. The first flexible portion 64 comes into close contact with an inner surface of the coupling member 110 and is secured to the coupling member 110. The first flexible portion 64 may be interference-fitted to the coupling member 110.

The rotating member 120 has a hollow cylindrical shape such that the coupling member 110 and the second flexible portion 65 are inserted through the rotating member 120. A rear end of the third flexible portion 66 is secured to the rotating member 120. A rear end of the rotating member 120 is axially rotatably coupled to the coupling member 110 and the flexible member 60. The coupling member 110 is circumferentially provided at an outer surface thereof with rotation guide bosses 112, and the rotating member 120 is provided at an inner surface thereof with rotation guide recesses 121 corresponding to the rotation guide bosses 112. The rotating member 120 is slidable and rotatable relative to the coupling member 110 in a state in which the rotating guide bosses 121 are inserted in the rotation guide recesses 112.

The rotating member 120 includes a hollow cylindrical rotating body 130 secured to a rear end of the third flexible portion 66 so as to be rotated along with the third flexible portion 66, and a rotating disc 140 secured to a rear end of the rotating body 130 so as to receive an external force. To secure the rear end of the third flexible portion 66 to the rotating body 130, a contact member 131 is placed on a front end of the rotating body 130. The rotating disc 140 has a circular disc shape and may be provided throughout an outer periphery thereof with rotating teeth 141. The rotating body 130 and the rotating disc 140 may be separately provided so as to be coupled with each other, or may be integrally formed with each other.

If the operator applies axial torque to the rotating member 120, the second flexible portion 65 exhibits torsional motion and the third flexible portion 66 is axially rotated along with the rotating member 120. When torque applied to the rotating member 120 is released, the second flexible portion 65 may be returned to an original state thereof by torsional elastic energy accumulated in the second flexible portion 65. Thus, to prevent the second flexible portion 65 from being returned to an original state before torsional deformation when the torque applied to the rotating member 120 is released, the friction member 150 is placed between the coupling member 110 and the rotating member 120 to increase friction between the coupling member 110 and the rotating member 120. The friction member 150 includes an elastic member 151 to come into contact with the coupling member 110 and the rotating member 120 with elastic energy accumulated therein. One end of the elastic member 151 is secured to the coupling member 110 and the other end of the elastic member 151 is positioned to apply elastic force to the rotating member 120.

Hereinafter, operation of the torsional device 100 will be described. As illustrated in FIG. 7, the operating unit 50 includes a housing 500 defining an external appearance of the operating unit 50, and the bending operation unit 51 and a rotation operation unit 55 mounted to the housing 500.

The bending operation unit 51 includes a bending operation knob 54 placed at an outer surface of the housing 500 and the bending wire 52 connected to the bending operation knob 54. A power transmission member, such as a bending operation pulley 53, may be placed between the bending operation knob 54 and the bending wire 52. A power source, such as an electrically driven motor, may be additionally provided between the bending operation knob 54 and the power transmission member.

The rotation operation unit 55 includes a rotation operation knob 59 provided at the outer surface of the housing 500, and a power transmission member, such as a gear assembly 57, placed in the housing 500 to transmit torque that the operator applies to the rotation operation knob 59 to the torsional device 100. The power transmission member may include a wire and pulley, or a chain and sprocket, in addition to the gear assembly 57. Also, a power source, such as an electrically driven motor, may be additionally provided between the rotation operation knob 59 and the gear assembly 57.

One side of the gear assembly 57 is connected to the rotation operation knob 59 and the other side of the gear assembly 57 is connected to the rotating disc 140 of the torsional device 100. The torque that the operator applies to the rotation operation knob 59 is transmitted to the rotating member 120 of the torsional device 100 via the gear assembly 57. If the rotating member 120 of the torsional device 100 applies axial torque to the rear end of the third flexible portion 66, the second flexible portion 65 exhibits torsional motion because movement of the first flexible portion 64 is restricted by the coupling member 120. Thereby, the third flexible portion 66 is rotated along with the rotating member 120 of the torsional device 100 and the bending member 70 connected to the front end of the flexible member 60 as illustrated in FIG. 2 is axially rotated along with the third flexible portion 66.

If torque applied to the rotation operation knob 59 is released, torsional elastic energy accumulated in the second flexible portion 65 acts as restoration force to return the second flexible portion 65 to an original state before torsional deformation. However, since the friction member 150 secured to the coupling member 110 applies friction corresponding to the restoration force to the rotating member 120, the second flexible portion 65 is not returned to an original state before deformation and the rotating member 120 can be continuously rotated.

### MODE FOR INVENTION

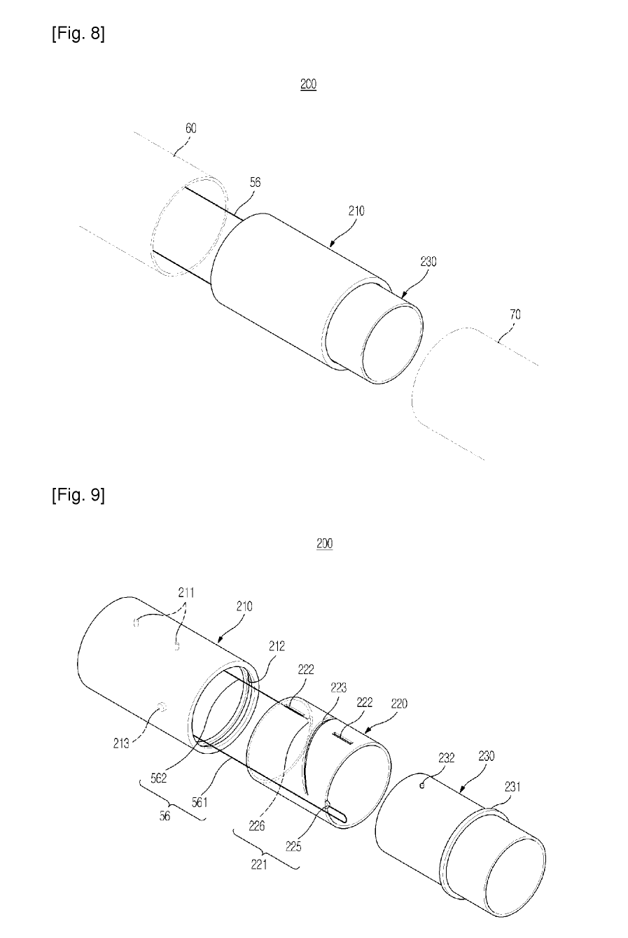

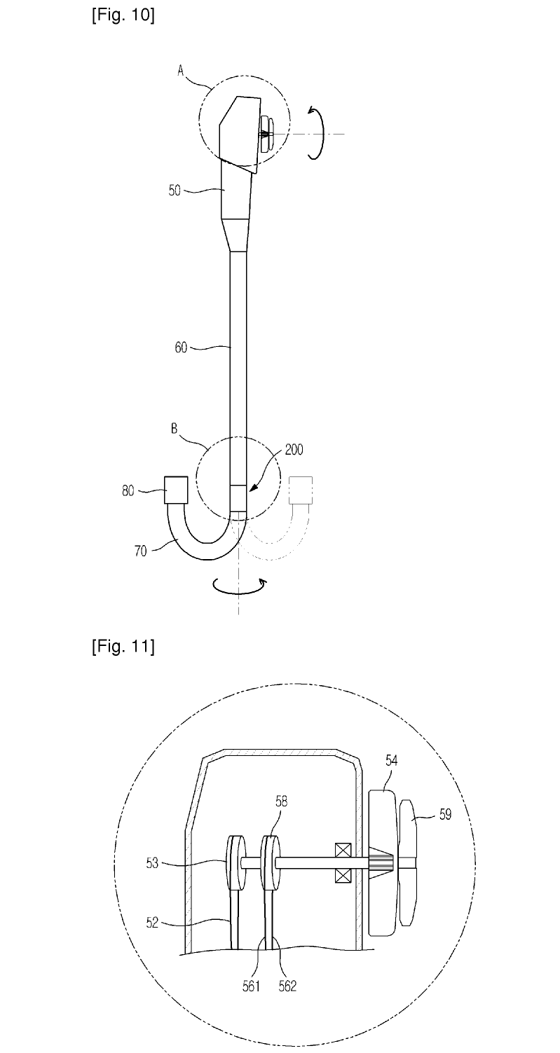

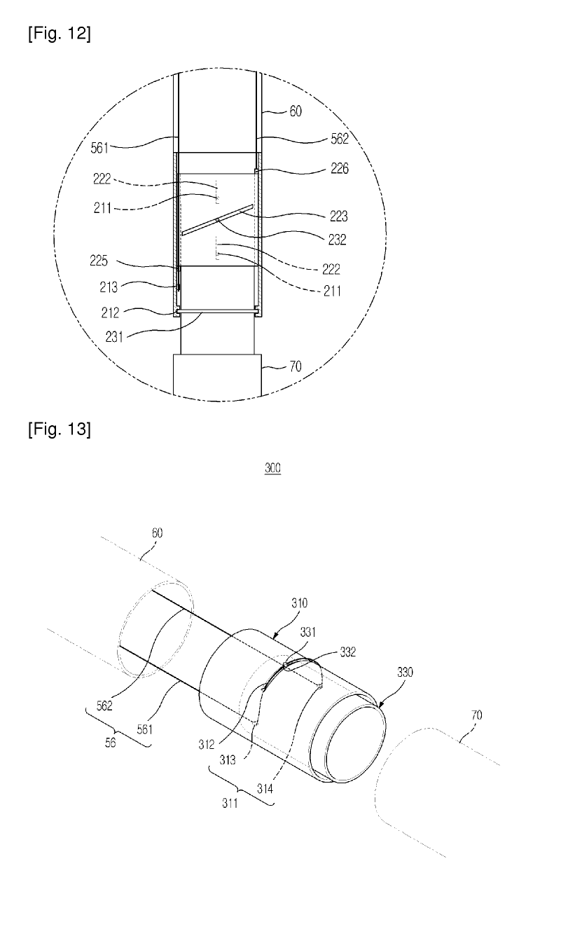

FIG. 8 is a perspective view illustrating a rotating device according to another embodiment of the present invention, FIG. 9 is an exploded perspective view illustrating major components of the rotating device, and FIGS. 10 to 12 are views illustrating an operation of the rotating device.

As illustrated in FIGS. 8 to 12, the rotating device includes a rotating tube assembly 200 formed by axially connecting three tubes to one another.

Both ends of the rotating tube assembly 200 may be connected to the flexible member 60. However, since the entire rotating tube assembly 200 is rigid, connecting both ends of the rotating tube assembly 200 to the flexible member 60 may deteriorate the ability of the flexible member 60 to bend. Therefore, the rotating tube assembly 200 may be placed between the flexible member 60 and the bending member 70.

The rotating tube assembly 200 includes a stationary tube 210, a sliding tube 220 inserted into the stationary tube 210 so as to reciprocally move in a longitudinal direction within the stationary tube 210, and a rotating tube 230 axially rotatably coupled to the stationary tube 210. The stationary tube 210, the sliding tube 220 and the rotating tube 230 may be coupled to one another on the same axis.

A rear end of the stationary tube 210 is secured to the front end of the flexible member 60. The stationary tube 210 is circumferentially provided at an inner wall surface thereof with a rotation guide groove 212 to guide rotation of the rotating tube 230. The rotation guide groove 212 is formed near a front end of the stationary tube 210.

The stationary tube 210 includes reciprocation guide pins 211 protruding inward from the inner wall surface of the stationary tube 210 so as to guide reciprocation of the sliding tube 220. The stationary tube 210 further includes a rotation wire hanger 213. The rotation wire hanger 213 will be described later.

The sliding tube 220 has an outer diameter corresponding to an inner diameter of the stationary tube 210 and is inserted into the stationary tube 210. The sliding tube 220 includes reciprocation guide slots 222 longitudinally perforated in a wall thereof, into which the reciprocation guide pins 211 of the stationary tube 210 are respectively inserted.

Rotation wires 56 are connected to the sliding tube 220. The sliding tube 220 includes rotation wire connectors 221 to which the rotation wires 56 are connected. The rotation wires 56 include a first rotation wire 561 to move the sliding tube 220 rearward and a second rotation wire 562 to move the sliding tube 220 forward. The rotation wire connectors 221 include a first rotation wire connector 225 connected to the first rotation wire 561 and a second rotation wire connector 226 connected to the second rotation wire 562.

The first rotation wire connector 225 and the second rotation wire connector 226 are provided respectively at front and rear ends of the sliding tube 220. Also, the first rotation wire connector 225 and the second rotation wire connector 226 are located at symmetrical positions about a center axis of the sliding tube 220.

The rotation wire hanger 213 of the stationary tube 210 is located in front of the first rotation wire connector 225. The rotation wire hanger 213 and the first rotation wire connector 225 are aligned in a straight line in a longitudinal direction of the sliding tube 220. Thus, the first rotation wire 561 is first caught by the rotation wire hanger 213 of the stationary tube 210 and thereafter, is connected to the first rotation wire connector 225 of the sliding tube 220. As such, if tension is applied to the first rotation wire 561, the sliding tube 220 is moved forward of the stationary tube 210.

To reduce friction between the first rotation wire 561 and the rotation wire hanger 213, the rotation wire hanger 213 has a cylindrical shape. The rotation wire hanger 213 may include a roller (not shown) rotatably secured to the stationary tube 210.

During reciprocation of the sliding tube 220, the rotating tube 230 is axially rotated while being coupled to the sliding tube 220. To this end, the sliding tube 220 has a rotation leading slit 223 perforated in a curved circumferential wall thereof to extend obliquely relative to a center axis of the sliding tube 220. The rotation leading slit 223 is perforated in the sliding tube 220 to communicate the interior of the sliding tube 220 with the outside.

The rotating tube 230 includes a rotation protrusion 231 circumferentially arranged on an outer surface of the rotating tube 230. The rotation protrusion 231 is inserted into the rotation guide groove 212 of the stationary tube 210. Longitudinal movement of the rotating tube 230 is restricted by the rotation protrusion 231 inserted into the rotation guide groove 212.

The rotating tube 230 includes a rotation pin 232 protruding from the outer surface of the rotating tube 230. The rotation pin 232 is inserted into the rotation leading slit 223 of the sliding tube 220. The rotation pin 232 is moved along the rotation leading slit 223 during reciprocation of the sliding tube 220.

Hereinafter, operation of the rotating tube assembly 200 will be described. As illustrated in FIGS. 10 to 12, the operating unit 50 includes the housing 500 defining an external appearance of the operating unit 50, and the bending operation unit 51 and the rotation operation unit 55 mounted to the housing 500.

The bending operation unit 51 includes the bending operation knob 54 rotatably placed at the housing 500 to allow the user to grip and rotate the bending operation knob 54, a bending operation pulley 53 connected to the bending operation knob 54, and the bending wire 52 connected to the bending operation pulley 53.

The rotation operation unit 55 includes the rotation operation knob 59 rotatably provided at the housing 500 to allow the user to grip and rotate the rotation operation knob 59, a rotation operation pulley 58 connected to the rotation operation knob 59, and the rotation wires 56 connected to the rotation operation pulley 58. The bending operation pulley 53 and the rotation operation pulley 58 have the same axis of rotation.

The bending operation unit 51 and the rotation operation unit 55 may be operated independently of each other. Thus, the rotation operation knob 59 cannot be rotated while the operator rotates the bending operation knob 54 and on the contrary, the bending operation knob 54 cannot be not rotated while the operator rotates the rotation operation knob 59.

If the operator rotates the bending operation knob 54, tension is applied to the bending wire 52, causing the bending member 70 to be bent. However, the bending member 70 has only one degree of freedom and therefore, cannot move the view member of the endoscope 10 to various positions within the human body using only the bending operation unit 51.

If the operator rotates the rotation operation knob 59 in a given direction to apply tension to the first rotation wire 561, the sliding tube 220 is moved forwardly within the stationary tube 210. In this case, rotation of the sliding tube 220 is restricted relative to the stationary tube 210 because the reciprocation guide pins 211 of the stationary tube 210 are inserted into the reciprocation guide slots 222 of the sliding tube 220.

When the sliding tube 220 is moved forward, the rotation pin 232 of the rotating tube 230 is moved along the rotation leading slit 223. Forward or rearward movement of the rotating tube 230 is restricted by the rotation protrusion 231 of the rotating tube 230 inserted into the rotation guide groove 212 of the stationary tube 210. Thus, the rotating tube 230 is rotated in a given direction relative to the stationary tube 210 and the bending member 70 connected to the front end of the rotating tube 230 is rotated along with the rotating tube 230.

If the operator rotates the rotation operation knob 59 in an opposite direction, the rotating tube 230 and the bending member 70 are rotated in opposite directions by the above described operation.

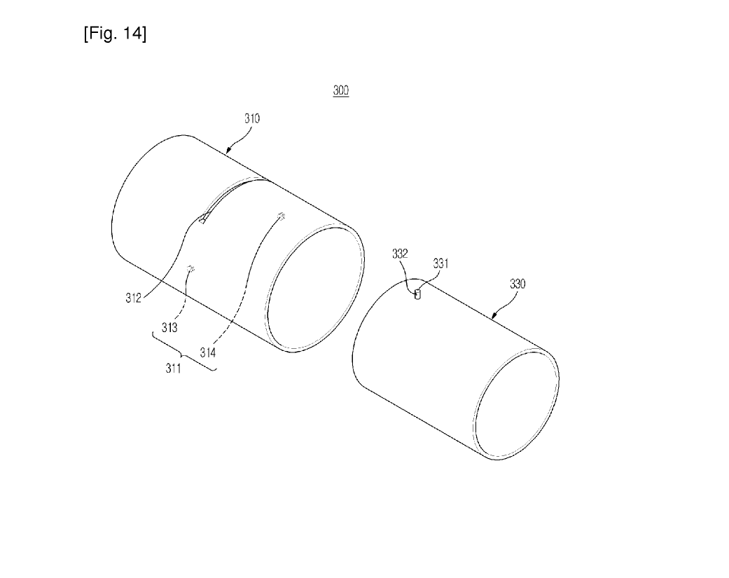

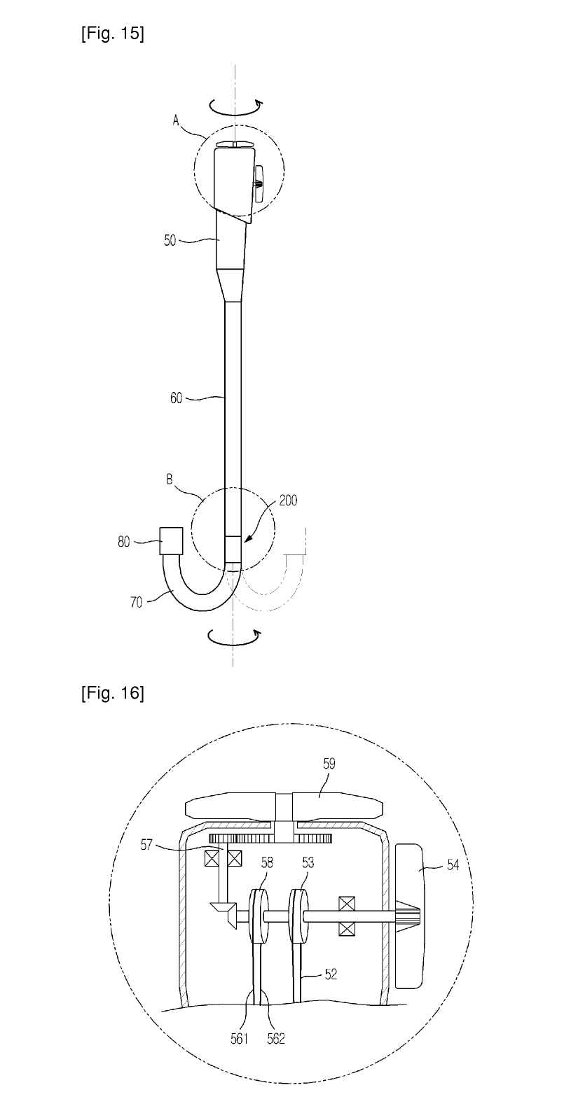

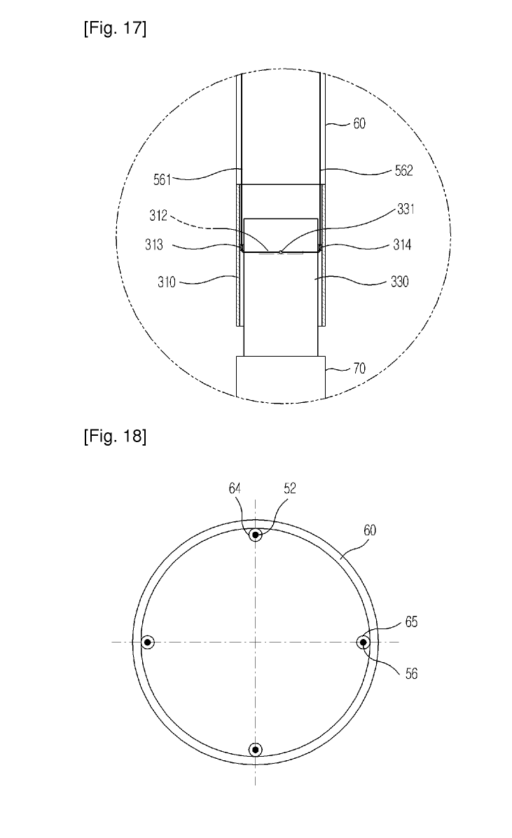

FIG. 13 is a perspective view illustrating a configuration of a rotating device according to a further embodiment of the present invention, FIG. 14 is an exploded perspective view illustrating major components of the rotating device, and FIGS. 15 to 17 are views illustrating an operation of the rotating device.

As illustrated in FIGS. 13 to 17, a rotating tube assembly 300, unlike the rotating tube assembly 200 illustrated in FIG. 9, is formed by axially connecting two tubes to each other.

The rotating tube assembly 300 includes a stationary tube 310 and a rotating tube 330 inserted into the stationary tube 310 so as to be rotated relative to the stationary tube 310.

The stationary tube 310 may be connected to the front end of the flexible member 60 and the rotating tube 330 may be connected to the rear end of the bending member 70. Thus, as the rotating tube 300 is axially rotated relative to the stationary tube 310, the bending member 70 is rotatable in an axial direction of the rotating tube assembly 300 relative to the flexible member 60.

The stationary tube 310 includes a rotation guide slit 312 perforated in a circumferential direction thereof. The rotating tube 330 includes an outwardly protruding rotation pin 331. The rotation pin 331 is configured to be inserted into the rotation guide slit 312 of the stationary tube 310. The rotating tube 330 is rotatable relative to the stationary tube 310 as the rotation pin 331 is moved along the rotation guide slit 312 of the stationary tube 310.

The rotation wire 56 is connected to the rotating tube 330. The rotation wire 56 includes the first rotation wire 561 to rotate the rotating tube 330 in a given direction and the second rotation wire 562 to rotate the rotating tube 330 in an opposite direction.

The rotating tube 330 includes rotation wire connectors 332 to which the rotation wires 56 are secured. The rotation wire connector 332 may be integrally formed with the rotation pin 331. The first rotation wire 561 and the second rotation wire 562 are connected to the rotation wire connectors 332 respectively.

The stationary tube 310 is provided at an inner wall surface thereof with rotation wire hangers 311 to guide the rotation wires 56 to the rotation wire connectors 332. The rotation wire hangers 311 include a first rotation wire hanger 313 to guide the first rotation wire 561 to the rotation wire connector 332 and a second rotation wire hanger 314 to guide the second rotation wire 562 to the rotation wire connector 332. The second rotation wire hanger 314 is located on a circumference of the rotating tube 330 where the first rotation wire hanger 313 is also located. That is, the first rotation wire hanger 313 and the second rotation wire hanger 314 are located on a single circumference. A straight line connecting the first rotation wire hanger 313 and the second rotation wire hanger 314 to each other may pass the center of the rotating tube 330.

The circumference of the rotating tube 330 where the rotation wire hangers 311 are located may be equal to or be adjacent to a circumference where the rotation guide slot 312 is perforated. The rotation wire hanger 312 may include a rotation roller (not shown) to reduce friction with the rotation wire 56.

Hereinafter, operation of the rotating tube assembly 300 will be described. As illustrated in FIGS. 15 to 17, the operating unit 50 includes the housing 500 defining an external appearance of the operating unit 50, and the bending operation unit 51 and the rotation operation unit 55 mounted to the housing 500.

The bending operation unit 51 includes the bending operation knob 54 rotatably placed at the housing 500 to allow the user to grip and rotate the bending operation knob 54, the bending operation pulley 53 connected to the bending operation knob 54, and the bending wire 52 connected to the bending operation pulley 53.

The rotation operation unit 55 includes the rotation operation knob 59 rotatably provided at the housing 500 to allow the user to grip and rotate the rotation operation knob 59, the rotation operation pulley 58 connected to the rotation operation knob 59, and the rotation wires 56 connected to the rotation operation pulley 58. The bending operation pulley 53 and the rotation operation pulley 58 have the same axis of rotation.

The rotation operation knob 59 and the bending operation knob 54 may have rotating shafts that are perpendicular to each other. The gear assembly 57 is placed between the rotation operation knob 59 and the rotation operation pulley 58.

If the operator rotates the rotation operation knob 59 in a given direction to apply tension to the first rotation wire 561, the first rotation wire 561 applies force to the rotation wire connector 332 of the rotating tube 330 in the given direction. The rotation pin 331 of the rotating tube 330 is moved along the rotation guide slit 312 in the given direction, and the rotating tube 330 is rotated by a predetermined angle corresponding to a distance by which the rotation pin 331 is moved. Thus, the bending member 70 connected to the rotation tube 330 is rotated along with the rotation tube 330 in an axial direction of the rotating tube 330.

If the operator rotates the rotation operation knob 59 in an opposite direction, the rotating tube 330 and the bending member 70 are rotated in opposite directions by the above described operation.

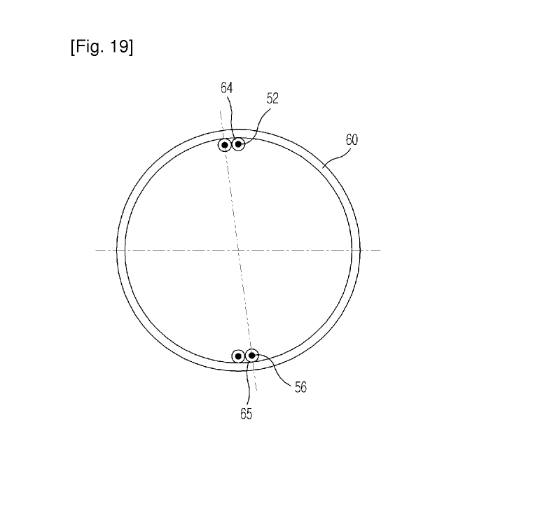

FIGS. 18 and 19 are views illustrating arrangement of the rotation wire according to an embodiment of the present invention.

As illustrated in FIGS. 18 and 19, a pair of rotation wire guide tubes 65 and a pair of bending wire guide tubes 64 are inserted into the flexible member 60. The rotation wire guide tubes 65 and bending wire guide tubes 64 are secured to the inner wall surface of the flexible member 60.

The rotation wires 56 and the bending wires 52 are respectively inserted into the rotation wire guide tubes 65 and the bending wire guide tubes 64. The pair of rotation wires 56 and the pair of bending wires 52 are arranged symmetrically about the center axis of the flexible member.

The rotation wires 56 and the bending wires 52 may be spaced apart from one another by arbitrary distances because they are independent of each other. As illustrated in FIGS. 18 and 19, the rotation wires 56 and the bending wires 52 may be spaced apart from one another by a possible maximum distance. Also, as illustrated in FIGS. 18 and 19, the rotation wire 56 and the bending wire 52 may be arranged close to each other.

### INDUSTRIAL APPLICABILITY

The present invention is applicable to a medical endoscope having a bending member for endoscopic inspection.

Although the embodiments of the present invention have been disclosed for illustrative purposes, those skilled in the art will appreciate that various modifications, additions and substitutions are possible, without departing from the scope and spirit of the invention as disclosed in the accompanying claims.

1. An endoscope comprising:
a flexible member which is freely bendable upon receiving external force;
a bending member provided at a front end of the flexible member, the bending member being bendable with one degree of freedom;
a rotating device rotatably coupled to the flexible member in an axial direction of the flexible member and serving to rotate the bending member in the axial direction; and
an operating unit connected to the rotating device and having a rotation operation unit to rotate the rotating device.

2. The endoscope according to claim 1, wherein the bending member includes a plurality of bending segments jointed to one another and elastic members placed between the respective bending segments to return the plurality of bending segments to original positions thereof.

3. The endoscope according to claim 1, wherein at least a part of the flexible member undergoes torsion when axial torque is applied to the flexible member, and
wherein the bending member is secured to the front end of the flexible member and is rotatable along with the flexible member.

4. The endoscope according to claim 3, wherein the rotating device includes a torsional device coupled to the flexible member so as to apply the axial torque to the flexible member, and
wherein the flexible member includes a first flexible portion secured to the operating unit, a second flexible portion connected to a front end of the first flexible portion so as to undergo torsion when the torque is applied thereto, and a third flexible portion connected to a front end of the second flexible portion so as to be rotated in the axial direction.

5. The endoscope according to claim 4, wherein the torsional device includes a coupling member secured to the operating unit and the first flexible portion so as to secure the first flexible portion to the operating unit, and a rotating member rotatably coupled to the coupling member while being secured to the third flexible portion.

6. The endo scope according to claim 5, wherein the coupling member includes a rotation guide boss formed on a circumference thereof, and
wherein the rotating member includes a rotation guide recess into which the rotation guide boss is inserted.

7. The endoscope according to claim 5, wherein the rotating member includes a rotating body secured to the third flexible portion to extend toward the first flexible portion, and a rotating disc secured to the rotating body and adapted to receive the torque from the rotation operation unit.

8. The endoscope according to claim 5, wherein the torsional device further includes a friction member placed between the coupling member and the rotating member to increase friction.

9. (canceled)

10. (canceled)

11. The endoscope according to claim 1, wherein the rotation operation unit includes a rotation wire connected to the rotating device,
wherein the rotating device includes a rotating tube assembly formed by axially connecting at least two tubes to each other, and
wherein a front end of the rotating tube assembly is axially rotatable relative to a rear end of the rotating tube assembly.

12. The endoscope according to claim 11, wherein the rotating tube assembly includes a stationary tube secured to the flexible member and a rotating tube rotatably inserted into the stationary tube while being secured to the bending member.

13. The endoscope according to claim 12, wherein the stationary tube includes a rotation guide slit perforated in a circumference thereof,
wherein the rotating tube includes a rotation pin movably inserted into the rotation guide slit, and
wherein the rotating tube is rotatable relative to the stationary tube as the rotation pin is moved along the rotation guide slit.

14. The endoscope according to claim 13, wherein the rotating tube further includes a rotation wire connector to which the rotation wire is secured.

15. The endoscope according to claim 14, wherein the rotation wire connector is secured to the rotation pin.

16. The endoscope according to claim 14, wherein the stationary tube further includes a rotation wire hanger to guide the rotation wire to the rotation wire connector.

17. (canceled)

18. (canceled)

19. The endoscope according to claim 12, wherein the rotating tube assembly further includes a sliding tube coupled to the stationary tube so as to reciprocate in an axial direction of the stationary tube.

20. The endoscope according to claim 19, wherein the stationary tube includes a reciprocation guide pin to guide reciprocation of the sliding tube, and
wherein the sliding tube includes a reciprocation guide slot into which the reciprocation guide pin is inserted.

21. The endoscope according to claim 19, wherein the sliding tube includes a rotation leading slit perforated in a curved circumferential wall thereof to extend obliquely relative to a center axis of the sliding tube,
wherein the rotating tube includes a rotation pin inserted into the rotation leading slit, and
wherein the rotating tube is rotated during reciprocation of the sliding tube as the rotation pin is moved along the rotation leading slit.

22. The endoscope according to claim 19, wherein the stationary tube further includes a rotation guide groove to guide rotation of the rotating tube, and
wherein the rotating tube further includes a rotation protrusion circumferentially formed and inserted into the rotation guide groove.

23. An endoscope comprising:
a flexible member that is bendable in an arbitrary direction;
a bending member connected to a front end of the flexible member, the bending member being bendable with one degree of freedom;
an operating unit for an operator to grip placed behind the flexible member; and
a rotating device coupled to the flexible member so as to be rotated in an axial direction of the flexible member and serving to rotate the bending member in the axial direction,
wherein the operating unit includes a housing defining an external appearance of the operating unit, a bending operation unit mounted to the housing and connected to the bending member to enable bending of the bending member, and a rotation operation unit mounted to the housing and connected to the rotating device, and
wherein the bending operation unit includes a bending wire connected to the bending member.

24. The endoscope according to claim 23, wherein a rear end of the flexible member is secured to an inner position of the housing,
wherein the rotating device includes a torsional device coupled to the flexible member to surround at least a part of the flexible member so as to transmit axial torque to the flexible member, and
wherein the torsional device is placed within the housing.

25. The endoscope according to claim 23, wherein the bending operation unit further includes a bending operation pulley placed within the housing to operate the bending wire,
wherein the rotation operation unit includes a rotation wire connected to the rotating device and a rotation operation pulley placed within the housing to operate the rotation wire, and
wherein the bending operation pulley and the rotation operation pulley have the same axis of rotation.

26. The endoscope according to claim 23, wherein the bending operation unit further includes a bending operation knob rotatably mounted to the housing to operate the bending wire,
wherein the rotation operation unit includes a rotation wire connected to the rotating device and a rotation operation knob rotatably mounted to the housing to operate the rotation wire, and
wherein an axis of rotation of the bending operation unit and an axis of rotation of the rotation operation knob are perpendicular to each other.

27. The endoscope according to claim 23, wherein the rotation operation unit includes a rotation wire connected to the rotating device, and
wherein the bending wire and the rotation wire are arranged close to each other within the flexible member.

28. An endoscope comprising:
a flexible member that is bendable in an arbitrary direction;
a bending member connected to a front end of the flexible member, the bending member being bendable with one degree of freedom;
a torsional device coupled to the flexible member to transmit axial torque to the flexible member; and
an operating unit including a rotation operation unit connected to the torsional device,
wherein the flexible member includes a first flexible portion secured to the operating unit, a second flexible portion connected to a front end of the first flexible portion so as to undergo torsion when the torque is applied thereto, and a third flexible portion connected to a front end of the second flexible portion so as to be rotated in the axial direction, and
wherein the torsional device includes a coupling member secured to the operating unit and the first flexible portion so as to secure the first flexible portion to the operating unit, and a rotating member rotatably coupled to the coupling member while being secured to the third flexible portion.

29. (canceled)
